(19) United States
(12) Patent Application Publication
(10) Pub. No.: US 2022/0395169 A1
(43) Pub. Date: Dec. 15, 2022

(54) CANNULA CLEANING BRUSH AND RESERVOIR

(71) Applicants: Cory Allen Gaiser, Cincinnati, OH (US); Jonathan N. Gates, Flint, MI (US)

(72) Inventors: Cory Allen Gaiser, Cincinnati, OH (US); Jonathan N. Gates, Flint, MI (US)

(21) Appl. No.: 17/468,292

(22) Filed: Sep. 7, 2021

Related U.S. Application Data
(60) Provisional application No. 63/209,418, filed on Jun. 11, 2021.

(51) Int. Cl. A61B 1/12; A61B 90/70

(52) U.S. Cl. CPC A61B 1/122; A61B 1/125; A61B 90/70; A61B 2090/701

(57) ABSTRACT

The present invention discloses a cleaning device for a medical implement. The device comprises a receptacle member containing a cleaning agent and a detachable hollow member. The hollow member is sized for inserting within the receptacle member and at least partially submerged in the cleaning agent. The hollow member having a porous body portion adapted to receive a medical implement, for example, a cannula, and a plurality of bristle clusters arranged within the body portion adapted to engage the medical implement. The hollow member is configured to allow the medical implement to move freely and axially in engagement with bristle clusters to clean and mechanically dislodge any debris therefrom. The cleaning device further facilitates to perform an aspiration function of the cannula to draw sterile liquid through an inner portion of the cannula to flush and remove debris within the cannula.

### CROSS-REFERENCE TO RELATED APPLICATIONS

This application claims the benefit of priority under 35 U.S.C. 119(e) to U.S. Patent Application Provisional Application Ser. No. 63/209,418, entitled “CANNULA CLEANING BRUSH-TUBE AND RESERVOIR,” filed Jun. 11, 2021, the contents of which are hereby incorporated by reference in their entirety for any purpose.

### TECHNICAL FIELD

The present disclosure relates generally to a cleaning device for a medical implement, and more particularly, a cleaning device for a medical implement, for example, a cannula.

### BACKGROUND

Medical procedures which use endoscopes, and other elongated instruments which are inserted through surgical openings, are relatively expensive products and must be used multiple times. Accordingly, such devices of necessity must be cleaned and sterilized repeatedly. Such instruments typically include long narrow cannulas through which surgical implements and other devices are passed in a surgical procedure. The cannulas and lumens of such cannulas are subject to contamination by bodily fluids and materials and must be cleaned carefully before reuse. If they are not thoroughly cleaned prior to disinfection and sterilization, surgical debris can be passed to another patient leading to infection or other complications. Accordingly, it is very important to adequately clean the interiors of endoscopes and similar surgical instruments, which are often difficult to access. There is also a need to clean such instruments during medical procedures, as fluid and debris can obstruct the lumen. To date, no such “intra-operative” cleaning device is known.

Various techniques or devices have been previously proposed for cleaning the cannulas of the endoscopes, the simplest of which involves immersing the devices in solutions containing a detergent and/or an enzyme. Other applications use a small brush, constructed much like the conventional bottle brush having bristles locked between twisted wires, to reach the interior lumen of the cannula. Such brushes are not entirely effective as they do not carry the cleaning or enzymatic solution to the wall surfaces of the lumen. In addition, the bristles are liable to scratch or damage the interior surfaces of the endoscopes and leave hardened deposits thereon.

Generally, the cleaning operation is performed at the end of a medical procedure, under non-sterile conditions, and requires the cannula to be detached from its handle. The brush is inserted inside the cannula opening and moved in a back-and-forth motion to remove debris from the inside of the cannula, often while submerged in water or other cleaning solution. The removal of the cannula from its handle, in an attempt to clean internal and external debris, can create cross-contamination and is difficult to perform under sterile conditions. This also increased the likelihood of dropping the cannula, or its associated tools, thus contaminating these devices. When debris accumulates at the external opening of a cannula during a procedure it is typically removed manually with a towel or ridged instrument, this too increases the potential for cross-contamination.

Therefore, there is a need for a cleaning device for cleaning a medical implement. Further, there is a need for a cleaning device without much contact with the medical implement to avoid contamination. Further, there is a need for a cleaning device that enables the cleaning of the interior surfaces of the cannula without using bristles.

### SUMMARY OF THE INVENTION

The present invention discloses a cleaning device for a medical implement. The device comprises a receptacle member containing a cleaning agent and a detachable hollow member. The hollow member is sized for inserting within the receptacle member and at least partially submerged in the cleaning agent. The hollow member having a porous body portion adapted to receive a medical implement, for example, a cannula and a plurality of bristle clusters arranged within the body portion adapted to engage the medical implement. The hollow member is configured to allow the medical implement to move freely and axially in engagement with bristle clusters to clean and mechanically dislodge any debris therefrom. The cleaning device further facilitates to perform an aspiration function of a cannula to draw sterile liquid through an inner portion of the cannula to flush and remove debris within the cannula.

In one embodiment, the receptacle member is formed of a bottom wall, and a surrounding wall extending upwardly from a periphery of the bottom wall and cooperating with the bottom wall to define a space. The surrounding wall having an upper end opposite to the bottom wall.

In one embodiment, the body portion is composed of at least three cylindrical portions. At least one cylindrical portion is integrally connected to an adjacent cylindrical portion through at least one connector portion. The body portion having a first open end and an opposing second open end. The first open end having a cap member adapted to rest on an upper end of the receptacle member and the opposing second open-end rests proximal to the bottom wall of the receptacle member. The hollow member comprises a first cap member having an opening to receive the medical implement therethrough and defines the first open end.

The opposing second open end facilitates the cleaning agent to pass there through and at least partially fills the cleaning agent within the hollow member. Further, the body portion comprises a porous surface that facilitates the cleaning agent to pass there through and at least partially fills the cleaning agent within the hollow member. The cleaning device further comprises a removable seal adapted to cover the opening of the hollow member. The device further comprises a second cap member configured to lock with the first cap member to maintain the first cap member in a sterile condition.

### BRIEF DESCRIPTION OF THE DRAWINGS

FIG. 1 exemplarily illustrates a perspective view of a cleaning device according to an embodiment of the present invention.

FIG. 2 exemplarily illustrates an exploded view of a cleaning device of FIG. 1.

FIG. 3 exemplarily illustrates a perspective view of a hollow member of the cleaning device of FIG. 1.

FIG. 4 exemplarily illustrates a top perspective view of the hollow member of the cleaning device of the FIG. 1.

FIG. 5 exemplarily illustrates a cross-sectional view of the hollow member of the cleaning device of the FIG. 1.

FIG. 6 exemplarily illustrates an exploded view of a first cap member and a second cap member of the cleaning device of the FIG. 1.

FIG. 7 exemplarily illustrates a cannula received within the hollow member of the cleaning device of FIG. 1.

FIG. 8 exemplarily illustrates the cannula received within the cleaning device of FIG. 1.

### DETAILED DESCRIPTION OF EXAMPLE EMBODIMENTS

Example embodiments of the disclosure now will be described more fully hereinafter with reference to the accompanying drawings, in which example embodiments are shown. The concepts discussed herein may, however, be embodied in many different forms and should not be construed as limited to the example embodiments set forth herein; rather, these embodiments are provided so that this disclosure will be thorough and complete, and will fully convey the scope to those of ordinary skill in the art. Like numbers refer to like elements but not necessarily the same or identical elements throughout.

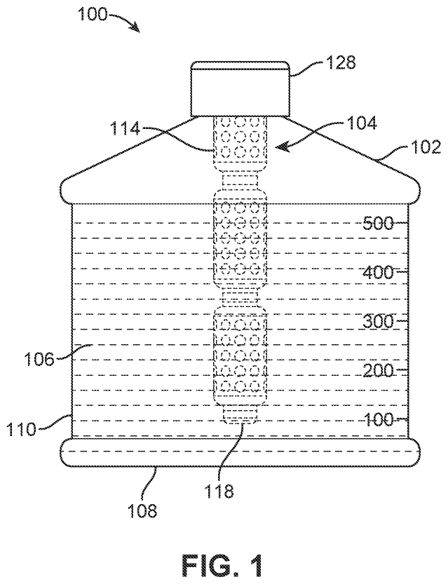

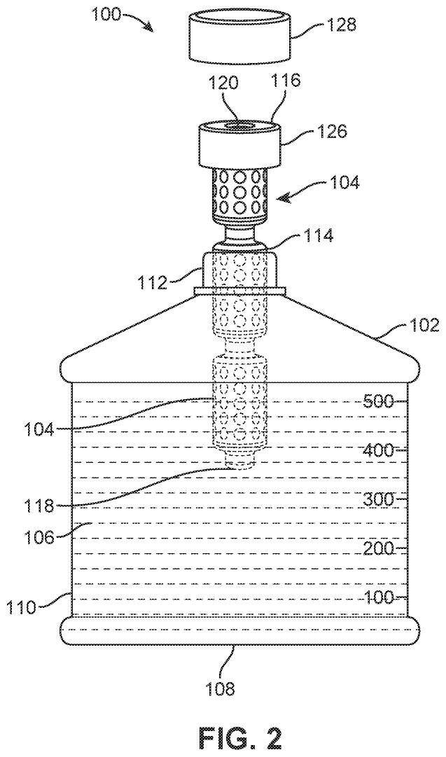

The present invention discloses a cleaning device 100 for a medical implement. FIG. 1 exemplarily illustrates a perspective view of a cleaning device 100 according to an embodiment of the present invention. FIG. 2 exemplarily illustrates an exploded view of the cleaning device 100 of FIG. 1. Referring to FIG. 1 and FIG. 2, the device 100 comprises a receptacle member 102 containing a cleaning agent 106 and a detachable hollow member 104. The hollow member 104 is sized for inserting within the receptacle member 102 and at least partially submerged in the cleaning agent 106. The hollow member 104 having a porous body portion 114 adapted to receive a medical implement and a plurality of bristle clusters 124 arranged within the body portion 114 adapted to engage the medical implement. The hollow member 104 is configured to allow the medical implement to move freely and axially in engagement with bristle clusters 124 to clean and mechanically dislodge any debris therefrom.

The cleaning agent 106 can be any chemical, substance or material that cleans the site of bacterial or even viral microorganisms, or any carrier that contains such chemical, substance or material. Examples of the cleaning agent 106 include isopropyl alcohol, chlorhexidine, povidone-iodine, hydrogen peroxide, soap, and hydrochloric acid as well as sodium chloride for “intra-operative” use. The term “medical implement” is used to denote any tool or object that can be used in a medical setting. Examples of medical implements include, but are not limited to, cannulas, access ports on tubing sets, for example, IV sets, access ports on catheters and other components or devices whereby regular cleaning is desired.

Referring to FIG. 2, the receptacle member 102 is formed by a bottom wall 108, and a surrounding wall 110 extending upwardly from a periphery of the bottom wall 108 and cooperating with the bottom wall 108 to define a receptacle space. The surrounding wall 110 has an upper end 112 opposite to the bottom wall 108. In one embodiment, the receptacle member 102 is a single-use receptacle member. In an embodiment, the bottom wall 108 of the receptacle member 102 may be wide to provide stability to the receptacle member 102. In one embodiment, the receptacle member 102 may be made of any suitable material, including but not limited to, translucent plastic. In one embodiment, the volume of the receptacle member 102 may be sufficient, for example 500 ml, to allow cleaning of multiple cannulas, for example, during a medical procedure.

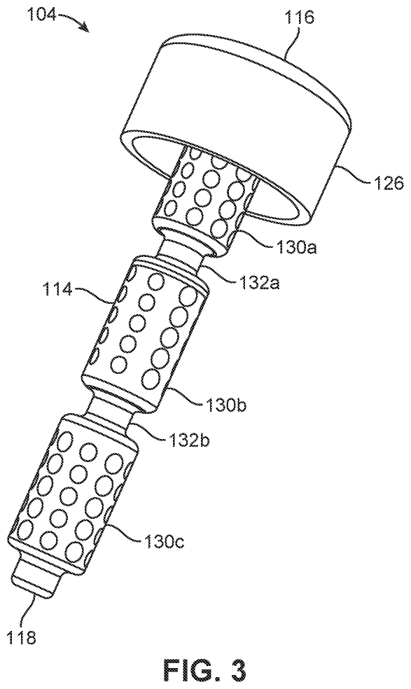

Referring to FIG. 3 to FIG. 7, the hollow member 104 is formed of the porous body portion 114 having a first open end 116 and an opposing second open end 118. The first open end 116 comprises a first cap member 126 that is adapted to rest on the upper end 112 of the receptacle member 102 and the second open end 118 rests proximal to the bottom wall 108 of the receptacle member 102, when the hollow member 104 is disposed within the receptacle member 102. The hollow member 104 further comprises on opening 120 formed on the first cap member 126 which defines the first open end 116 of the hollow member 104. In an example, the opening 120 may be a chamfered opening.

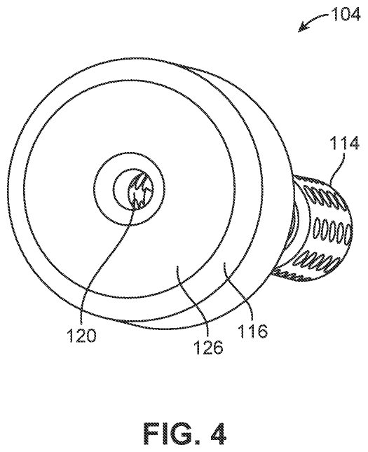

Referring to FIG. 3, the body portion 114 is composed of at least three cylindrical portions (130a, 130b, 130c) including a first cylinder portion 130a, a second cylinder portion 130b and a third cylinder portion 130c. At least one cylindrical portion (130a, 130b, 130c) is connected to an adjacent cylindrical portion (130a, 130b, 130c) through at least one connector portion (132a, 132b). For example, the first cylinder portion 130a is integrally connected to the second cylinder portion 132b through a first connector portion 132a. The second cylinder portion 132b is integrally connected to the third cylinder portion 132c through a second connector portion 132b. In one embodiment, the body portion 114 may be a single cylindrical portion. In another embodiment, the body portion 114 may have any geometrical shape. In one embodiment, the hollow member 104 maybe made of any suitable material, including but not limited to, plastic material and translucent plastic. FIG. 4 illustrates a top perspective view of the hollow member 104 of the cleaning device 100 of the FIG. 1. FIG. 4 further shows the opening 120 of the first cap member 126 formed at the first end 116 of the body portion 114.

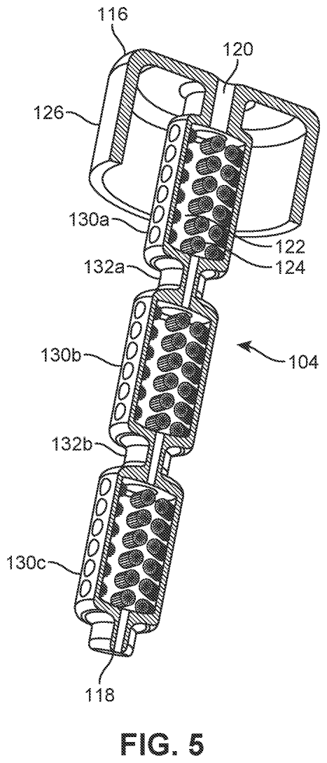

Referring to FIG. 5, the opening 120 is connected with a space 122 of the hollow member 104 to receive at least one medical implement there through. The space is further connected to the second open end 118 to allow passage of the cleaning agent 106 there through and at least partially fill the space with the cleaning agent 106. Additionally, the porous body portion 114 allows passage of cleaning agent 106 within hollow member 104 and at least partially fills the space with the cleaning agent 106. In one embodiment, the opening 120 is formed at the center of the first cap member 126. In another embodiment, the opening 120 may be formed position suitable for insertion of the medical implement.

Referring to FIG. 5, the plurality of bristle clusters 124 extends with the body portion 114. Each cluster 124 is formed of a number of bristles. The bristle cluster 124 may be arranged in a variety of different configurations, but will preferably be formed radially and extends towards an axis of the hollow member 104 at any orientation. The bristles may be made of any material. In some embodiments, the bristle is made of polymer material.

The cleaning device 100 further comprises a removable seal adapted to cover the opening 120 at the first open end 116 of the hollow member 104. The seal is provided to prevent spillage of the cleaning agent 106 from the receptacle member 102. The seal further maintains the sterile condition of the environment within the hollow member 104 and the receptacle member 102. The seal may be removed for passing the medical implement there through. In an embodiment, the seal is a foil-based seal. In another embodiment, the seal may be made of any other suitable material. The seal is attached to the opening 120 by glue, solvent, thermal bonding or any other suitable means.

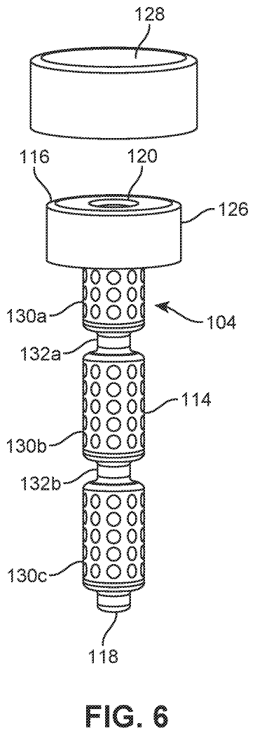

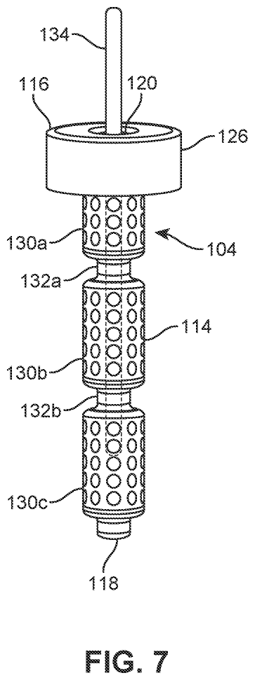

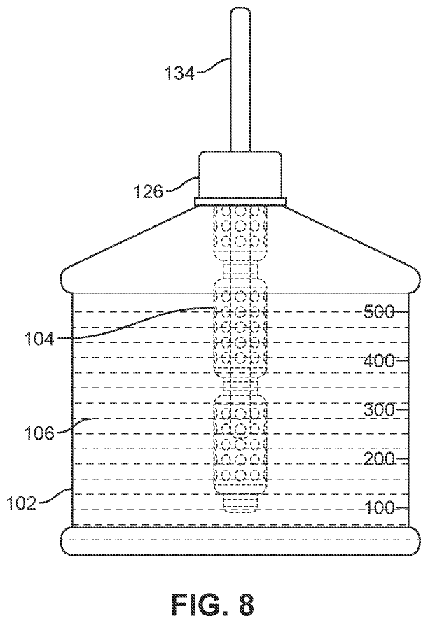

Referring to FIG. 6, the cleaning device 100 further comprises a second cap member 128 adapted to enclose and lock with the first cap member 126. In an embodiment, an exterior surface of the first cap member 126 may have one or more threads that are adapted to lock with one or more threads on an interior surface of the first cap member 126. In another embodiment, the first cap member 126 may snap-fit lock with the second cap member 128. In yet another embodiment, the first cap member 126 may lock with the second cap member 128 utilizing any conventional mechanisms. The first cap member 126 is maintained in sterile condition by the second cap member 128. The second cap member 128 is further configured to protect the seal from damage and contamination. Referring to FIG. 7, the opening 120 adapted to allow passage of a medical implement within the hollow member 104, for example, a cannula 134. In one embodiment, hollow member 104 may be provided as a separate unit and may be fixed to the receptacle member 102 while using the device 100. Referring to FIG. 8, the hollow member 104 is directly fixed to the receptacle member 102 and the cannula 134 is received within the hollow member 104 submerged within the cleaning agent 106.

During use, the second cap member 128 is removed from the first cap member 126, as shown in FIG. 6. Thereafter, the seal is removed from the first cap member 126 and the medical implement is passed through the opening 120 until a desired length of the medical implement is occupied within the hollow member 104, while maintaining sterility, as shown in FIG. 7 and FIG. 8. A back-and-forth motion or a linear motion is applied to the medical implement, for example, the cannula 134, allowing the bristle clusters 124 to mechanically remove the debris from an outer surface of the cannula 134. The user may also use an aspiration function of cannula 134, if available, to draw sterile liquid through an inner portion of cannula 134 allowing for flushing and removal of debris from the cannula 134. Once the cannula 134 is cleaned, the user can resume the use of cannula 134 for procedure in a sterile fashion. Thereby the present invention cleans a medical implement, for example, the cannula 134 in a sterile manner and facilitates to flush the cannula 134 with the cleaning agent 106.

The cleaning device 100 disclosed herein can also be designed to be disposed of after a single use, or it can be designed to be used multiple times. In either case, however, the cleaning device 100 can be reconditioned for reuse after at least one use. Reconditioning can include any combination of the steps of disassembly of the cleaning device 100, followed by cleaning or replacement of particular pieces and subsequent reassembly. In particular, the cleaning device 100 can be disassembled, and any number of the particular pieces or parts of the cleaning device 100 can be selectively replaced or removed in any combination. Those skilled in the art will appreciate that reconditioning of a cleaning device 100 can utilize a variety of techniques for disassembly, cleaning/replacement, and reassembly. Use of such techniques, and the resulting reconditioned cleaning device 100, are all within the scope of the present application.

In another aspect, a method for cleaning a cannula and distal end of a viewing scope is disclosed herein, comprising the steps of providing a cleaning device 100 having a receptacle member containing a cleaning agent and a detachable hollow member. The hollow member is sized for inserting within the receptacle member and at least partially submerged in the cleaning agent. The hollow member having a porous body portion adapted to receive a medical implement, for example, a cannula, and a plurality of bristle clusters arranged within the body portion adapted to engage the medical implement; inserting the cannula or viewing scope into the hollow member of the cleaning device 100 to thus scrub any bodily debris (e.g., fluid, such as blood or other bodily fluid and/or tissue debris) off of the cannula; and slidably reciprocating the distal end of the cannula and/or viewing scope with respect to the cleaning device 100 so that at least a portion engages the plurality of bristle clusters, thereby cleaning the cannula and/or viewing scope. In one embodiment, the plurality of bristle clusters may be immersed in a cleaning solution and used immediately for cleaning a medical instrument. In another embodiment, the cleaning solution is an enzymatic cleaner of the type which can degrade, disperse, or dissolve biological contaminant. Thus, and in accordance with one aspect of the teachings provided herein, a cannula and/or viewing scope may be cleared/cleaned of bodily tissue and/or fluid deposited within a cannula and/or on a lens of a viewing scope.

In still another aspect of the present disclosure, a kit can comprise in, in any combination, a cleaning device 100 configured in accordance with the teachings of the present disclosure, a trocar, and/or a cannula. In one example, the kit can include a cleaning device 100, and a corresponding cannula, as taught herein. In some embodiments, a cleaning fluid for cleaning a cannula and/or viewing scope is also included in the kit in order to preload the receptacle member of the cleaning device 100 before use, if so desired.

Although the features, functions, components, and parts have been described herein in accordance with the teachings of the present disclosure, the scope of coverage of this patent is not limited thereto. On the contrary, this patent covers all embodiments of the teachings of the disclosure that fairly fall within the scope of permissible equivalents.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

What is claimed is:

1. A cleaning device for a medical implement, comprising:
a receptacle member containing a cleaning agent, and
a removable hollow member sized for insertion within the receptacle member and at least partially submerged in the cleaning agent, wherein the hollow member having a porous body portion adapted to receive a medical implement and a plurality of bristle clusters arranged within the body portion adapted to engage the medical implement,
wherein the hollow member is configured to allow the medical implement to move freely and axially in engagement with bristle clusters to clean and mechanically dislodge any debris therefrom.

2. The cleaning device of claim 1, wherein the medical implement is a cannula.

3. The cleaning device of claim 2, facilitate to perform an aspiration function of cannula to draw cleaning agent through an inner portion of cannula allowing for flushing and removing the debris from the cannula.

4. The cleaning device of claim 1, wherein the receptacle member is formed by a bottom wall, and a surrounding wall extending upwardly from a periphery of the bottom wall and cooperating with the bottom wall to define a space, the surrounding wall having an upper end opposite to said bottom wall.

5. The cleaning device of claim 1, wherein the body portion having a first open end and an opposing second open end, wherein the first open end having a first cap member adapted to rest on the upper end of the receptacle member and the opposing second open-end rests proximal to the bottom wall of the receptacle member.

6. The cleaning device of claim 5, wherein the first cap member having a space to receive the medical implement there through and defines the first open end.

7. The cleaning device of claim 5, wherein the opposing second open end facilitates the cleaning agent to pass there through and at least partially fills the cleaning agent within the hollow member.

8. The cleaning device of claim 5, wherein the body portion comprises a porous surface that facilitates the cleaning agent to pass there through and at least partially fills the cleaning agent within the hollow member.

9. The cleaning device of claim 5, further comprises a removable seal adapted to cover the opening of the hollow member.

10. The cleaning device of claim 5, wherein the body portion is composed of at least three cylindrical portions, wherein at least one cylindrical portion is integrally connected to an adjacent cylindrical portion through at least one connector portion.

11. The cleaning device of claim 5, further comprises a second cap member configured to lock with the first cap member to maintain the first cap member in a sterile condition.
